(19) United States
(12) Patent Application Publication
(10) Pub. No.: US 2020/0025741 A1
(43) Pub. Date: Jan. 23, 2020

(54) METHOD TO PREDICT CROP NITROGEN STATUS USING REMOTE SENSING

(71) Applicant: Regents of the University of Minnesota, Minneapolis, MN (US)

(72) Inventors: Carl Rosen, Minneapolis, MN (US); Brian Bohman, Minneapolis, MN (US); David Mulla, Minneapolis, MN (US); Yuxin Miao, Minneapolis, MN (US)

(21) Appl. No.: 16/514,537

(22) Filed: Jul. 17, 2019

Related U.S. Application Data
(60) Provisional application No. 62/701,203, filed on Jul. 20, 2018.

(51) Int. Cl. G01N 33/24; G06K 9/00; G01N 21/27; G01N 33/00

(52) U.S. Cl. CPC G01N 33/24; G06K 9/00657; G01N 2033/245; G01N 33/0098; G01N 21/27

(57) ABSTRACT

A method of determining the nitrogen status of an area of land includes determining a critical nitrogen concentration for aboveground vegetation of plants in the area of land based on a dry weight biomass of entire plants and determining an actual nitrogen concentration for the aboveground vegetation of the plants. A critical nitrogen concentration for the entire plants is determined based on the dry weight biomass of the entire plants. The actual nitrogen concentration for the aboveground vegetation, the critical nitrogen concentration for the aboveground vegetation, the critical nitrogen concentration for the entire plants and the dry weight biomass of the entire plants are combined to form the nitrogen status for the area of land.

### CROSS-REFERENCE OF RELATED APPLICATION

The present application is based on and claims the benefit of U.S. provisional application Ser. No. 62/701,203, filed Jul. 20, 2018, the content of which is hereby incorporated by reference in its entirety.

### BACKGROUND

Nitrogen fertilizer applications are one of the most important management practices that affect plant yield. Insufficient nitrogen results in lower plant yield while over application of nitrogen can result in environmental impacts including contamination of surface water and groundwater, and greenhouse gas emissions. Inefficient nitrogen fertilizer applications are primarily the result of mismatched timing between the supply of nitrogen and plant nitrogen uptake. In-season nitrogen applications are most efficient when based on crop nitrogen status determined using plant tissue samples. However, plant tissue sampling is expensive, time consuming and can lack reliability.

### SUMMARY

A method of determining the nitrogen status of an area of land includes determining a critical nitrogen concentration for aboveground vegetation of plants in the area of land based on a dry weight biomass of entire plants and determining an actual nitrogen concentration for the aboveground vegetation of the plants. A critical nitrogen concentration for the entire plants is determined based on the dry weight biomass of the entire plants. The actual nitrogen concentration for the aboveground vegetation, the critical nitrogen concentration for the aboveground vegetation, the critical nitrogen concentration for the entire plants and the dry weight biomass of the entire plants are combined to form the nitrogen status for the area of land.

In accordance with a further embodiment, a computer includes a memory and a processor. The memory stores reflectance data for an area of a farm field and the processor executes instructions that use the reflectance data to determine an estimated nitrogen concentration for aboveground vegetation in the area and that use the estimated nitrogen concentration for aboveground vegetation in the area to determine a rate of nitrogen application needed by the area.

In accordance with a still further embodiment, a method includes receiving reflectance data for an area of a field and using the reflectance data to determine an estimated nitrogen concentration for aboveground vegetation in the area. The estimated nitrogen concentration for aboveground vegetation is then used to determine whether the area has an optimum amount of nitrogen.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 provides graphs of critical nitrogen concentrations as a function of dry weight biomass.

FIG. 2 provides a flow diagram of a method of determining crop nitrogen status in accordance with one embodiment.

FIG. 3 provides a block diagram of a system in accordance with one embodiment.

FIG. 4 provides a flow diagram of a method of determining biomass in accordance with one embodiment.

FIG. 5 provides a block diagram of elements used in a system in accordance with one embodiment.

### DETAILED DESCRIPTION OF ILLUSTRATIVE EMBODIMENTS

The present embodiments predict the need for in-season nitrogen fertilizer using a system of biophysical and physiological crop growth relationships which are parameterized using multispectral remote sensing. The embodiments use the sensed spectral signals to estimate the dry weight biomass (i.e., biomass without water) of entire plants and to estimate a nitrogen concentration of the aboveground vegetative portions of the plants. The estimated dry weight biomass and estimated nitrogen concentration are then used to identify a nitrogen application rate needed to reach an optimal nitrogen concentration for the entire plant or to identify an excess amount of nitrogen in the plants, and assess the resulting potential for negative environmental impacts.

The concentration of nitrogen in a portion of plant tissue is defined as the mass of the nitrogen contained in a portion of plant tissue divided by the dry weight biomass of that same portion of plant tissue. On any given day during the growth of the plant, there is a minimum nitrogen concentration that is necessary to maximize crop growth known as a critical nitrogen concentration. As a plant's dry weight biomass increases, the critical nitrogen concentration decreases. The relationship between the dry weight biomass of the entire plant or portions of the plant and the critical nitrogen concentration is often defined using a critical nitrogen dilution curve [CNDC], which defined as:

N_{c}=aW^{-b} Eq. b 1

where W is the dry weight biomass of the entire plant or a portion of the plant, N_{c} is the critical nitrogen concentration in the entire plant and a and b are parameters specific to each crop species, crop variety, and associated environmental conditions.

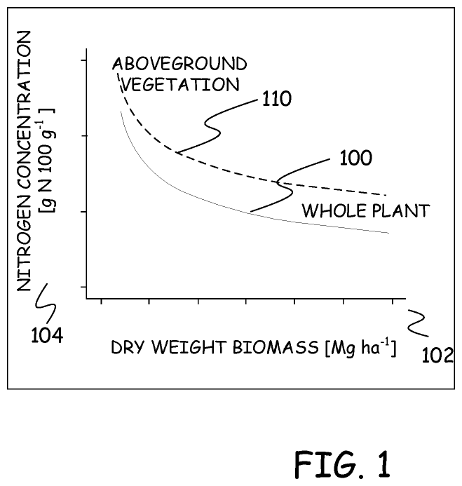

FIG. 1 shows a graph 100 of the relationship described in Eq. 1 between dry weight biomass and critical nitrogen concentration. In FIG. 1, dry weight biomass is shown along horizontal axis 102 and the critical nitrogen concentration is shown along vertical axis 104.

One measure of the deficiency or surplus of nitrogen in plants is the nitrogen nutrition index (NNI), which is defined as:

$$NNI = \frac{{N}_{a}}{{N}_{c}} \qquad Eq . 2$$

where Nₐ is the current nitrogen concentration and N_{c} is the critical nitrogen concentration. When the NNI is less than 1, there is a nitrogen deficiency, when the NNI is equal to 1 there is an optimal amount of nitrogen and when the NNI is greater than 1 there is an excess amount of nitrogen within the plant.

The NNI method is currently impractical for use in production systems due to the high labor costs of plant sampling and high laboratory analysis costs necessary to determine the dry weight biomass and current nitrogen levels in the plants.

In the various embodiments, multispectral remote sensing is used to predict aboveground vegetative nitrogen concentration and is used to predict the dry weight biomass of the entire plant using biophysical parameters such as canopy cover, leaf area index (LAI), and the aboveground vegetative biomass. The biophysical parameters related to the dry weight biomass of the entire plant are used in a growth model that predicts the dry weight biomass of the entire plant based on the biophysical parameters related to biomass of the entire plant and the crop growth conditions including solar radiation received by the plants and other climatic conditions. The estimated dry weight biomass is then used to predict a critical nitrogen concentration for the entire plant. The aboveground vegetative nitrogen concentration is used to determine an aboveground vegetative Nitrogen Nutrition Index (NNI_{V}) that is converted into an NNI for the entire plant. The difference between the NNI for the entire plant and an optimum NNI for the entire plant is then multiplied by the dry weight biomass and the critical nitrogen concentration for the entire plant to determine necessary nitrogen application rate or to quantify conditions of excessive nitrogen for the area of the field on a given date.

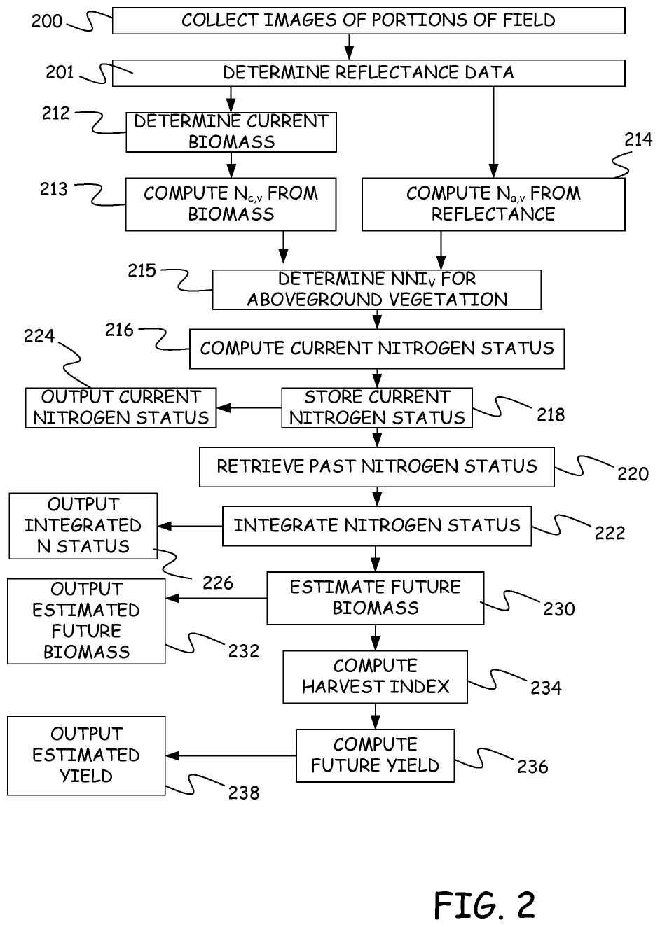

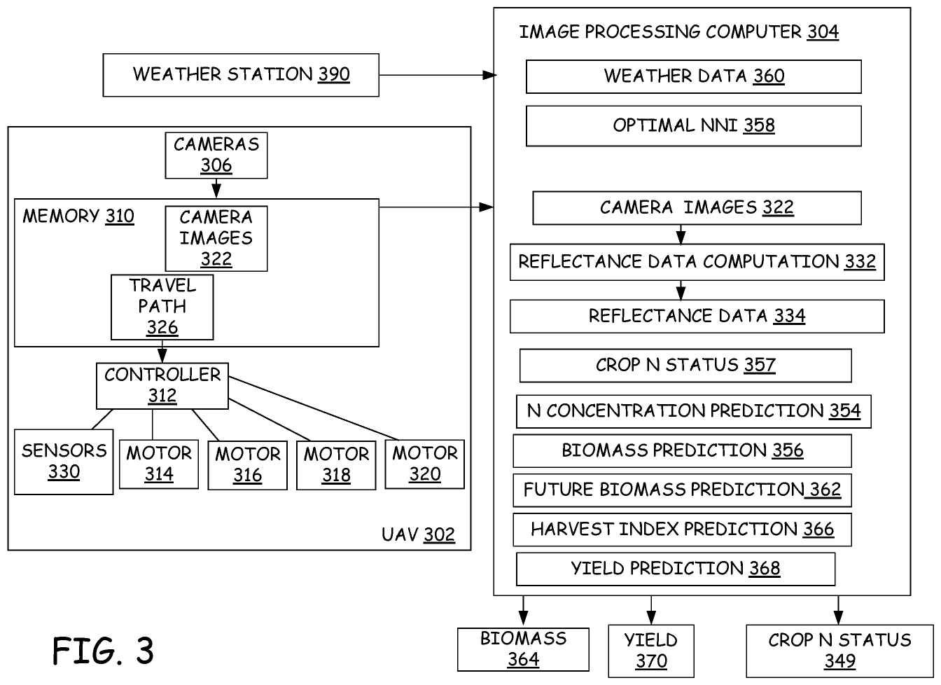

FIG. 2 provides a flow diagram of a method of determining and displaying the nitrogen status of an area of a field and FIG. 3 provides a block diagram of elements used to perform the steps of FIG. 2, in accordance with one embodiment.

In step 200 of FIG. 2, image data is collected by remote sensing instruments from the area of the field. Such remote sensing instruments can be mounted to ground, aerial, or satellite platforms, each having a unique set of advantages and limitations for use in precision agriculture. Ground-based sensors (e.g. CROPSCAN) are typically restricted to single point measurements and must be mounted to equipment that travels over the field area (e.g., tractors or irrigation equipment) to collect data over space. Aerial (e.g. MicaSense Altum) and satellite (e.g., PlanetLabs PlanetScope) platforms are better suited to capture image data, which contain thousands or millions of pixels per image and can efficiently collect data covering large areas. However, each platform has tradeoffs between spatial resolution, scalability to large areas, accuracy and consistency between sampling dates, number of spectral bands, and quality of spectral data.

In accordance with one embodiment, the reflectance data includes a spectral magnitude or light in the visible—near-infrared spectral regions (400-2500 nm) generally including bands in the blue (450-520 nm), green (520-600 nm), red (630-690 nm), red-edge (690-760 nm), and near-infrared (760-900 nm) spectral regions collected either as narrow bands or as broad bands and specifically including narrow bands at 460, 510, 560, 610, 660, 680, 710, 720, 740, 760, 810, 870, 950, 1320, 1500, 1720 nm. However, in other embodiments, other wavelengths are used.

In the embodiment of FIG. 3, the remote sensing instrument is a collection of cameras 306 mounted on an Unmanned Ariel Vehicle (UAV) 302, with each camera consisting of an array of sensors that are each capable of sensing light of a desired wavelength or band of wavelengths to form image data referred to as camera images 322. UAV 302 also includes a memory 310, a controller 312 and motors, such as motors 314, 316, 318 and 320. Camera images 322 from camera(s) 306 are stored in memory 310. A travel path 326 is also stored in memory 310 and represents the path that UAV 302 is to travel to capture images of a geographical area. In many embodiments, travel path 326 is a low altitude path. Travel path 326 is provided to controller 312, which controls motors 314, 316, 318 and 320 to drive propellers so that UAV 302 follows travel path 326. One or more sensors, such as sensors 330 provide feedback to controller 312 as to the current position of UAV 302 and/or the accelerations that UAV 302 is experiencing.

Periodically or in real-time, UAV 302 provides camera images 322 to image processing computer 304, which stores camera images 322 in a memory in computer 304. Images 322 may be provided over a wireless connection, a wired connection, or a combination of both between UAV 302 and image processing computer 304.

As noted above, camera images 322 may alternatively or additionally be provided by one or more satellites or by one or more ground-based sensors.

At step 201, camera images 322 are converted into reflectance data 334 by a reflectance data computation module 332 using parameters that are based on incident solar radiation (determined from an incident light sensor), from captured images of a calibrated reflectance panel that has known spectral properties, or based on atmospheric conditions.

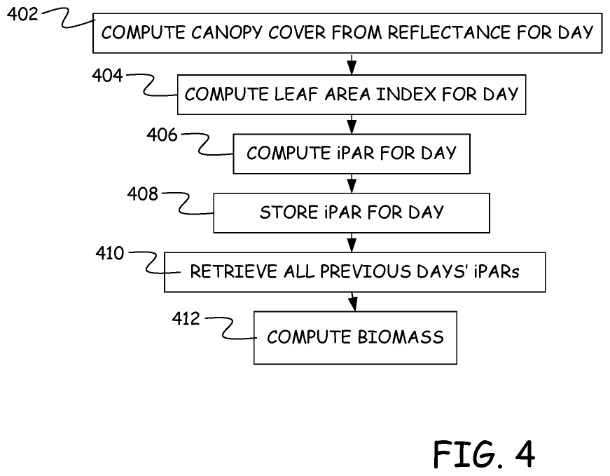

At step 212, a dry weight biomass is determined for an area captured in the images. In some embodiments, the dry weight biomass is determined by sampling plants in the area while in other embodiments, the dry weight biomass is estimated from the reflectance data. There are several ways to compute the dry weight biomass from reflectance data 334. FIG. 4 provides a flow diagram of one such method.

At step 402, a percentage of canopy cover is determined from the reflectance data for a current day by a processor in computer 304 executing a dry weight biomass prediction module 356. In accordance with one embodiment, the percent of canopy cover is calculated as:

CCⱼ=f(VI, Reflectance) Eq. 3

where Reflectance represents a subset of reflectance measurements 334 collated from the multiple spectral bands and VI represents a subset of vegetative indices able to be calculated using a given subset of spectral bands collected for a given set of imagery in reflectance data. For example, in one embodiment, the canopy cover is calculated as:

$${CC}_{j} = \frac{MSAVI {2}_{j} - MSAVI {2}_{CC ( 0 \% )}}{MSAVI {2}_{CC ( 100 \% )} - MSAVI {2}_{CC ( 0 \% )}} \qquad Eq . 4$$

where CCⱼ is the canopy cover on day j, MSAVI2ⱼ is calculated as:

$$MSAVI {2}_{J} = \frac{2 {R}_{NIR} + 1 - \sqrt{{( 2 {R}_{NIR} + 1 )}^{2} - 8 ( {R}_{NIR} - {R}_{R} )}}{2} \qquad Eq . 5$$

where R_{NIR} is the average magnitude of the reflectance in the near infrared range (760-900 nm) from all sensors across all images from the area of the field, and R_{R} is the average magnitude of reflectance in the red range (630-690 nm), MSAVI2 is the Modified Soil Adjusted Vegetation Index 2, MSAVI2ⱼ representing the index on day j, MSAVI2_{CC(0%)} represents the value of the index for bare soil and _{MSAVI}2_{CC(100%)} represents the value of the index for a full crop canopy. In equation 5, the full canopy cover and the bare soil values for the index are used to scale the index because MSAVI2 saturates at full canopy cover.

At step 404, the canopy cover is used by dry weight biomass prediction module 356 to compute a leaf area index (LAI) as:

LAIⱼ=f(CCⱼ) Eq. 6

where LAIⱼ is the leaf area index on day j and CCⱼ is the canopy cover on day j and where the function is dependent on the crop. For example, in one embodiment, the leaf area index is computed as:

LAIⱼ=3*(CCⱼ) Eq. 7

In other embodiments, the Leaf Area Index is computed directly from the reflectance data without determining the canopy cover first as:

LAIⱼ=f(VI, Reflectance) Eq. 8

where Reflectance represents a subset of reflectance measurements collated from the multiple spectral bands and VI represents a subset of vegetative indices able to be calculated using a given subset of spectral bands collected for a given set of imagery in reflectance data.

At step 406, the leaf area index is then used by dry weight biomass prediction module 356 to compute the amount of photosynthetically active radiation intercepted by the leaves of the plant (iPAR) using:

iPARⱼ=0.50*SRADⱼ*(1−e^{(-k*LAI}ⱼ)) Eq. 9

where iPARⱼ is the intercepted photosynthetically active radiation received on day j, SRADⱼ is the incident solar radiation received on day j as determined by a weather station 390 and provided as weather data 360 and k is an extinction co-efficient value, which in one embodiment ranges between 0.46 and 0.77.

At step 408, the calculated iPAR value for the current day is stored in memory and at step 410 all previous days' iPAR values are retrieved from memory.

At step 412, dry weight biomass prediction module 356 applies the iPAR values for the previous days and the current day to a growth model to estimate the dry weight biomass of the entire plant on the current day. Thus, this model estimates how much the plant will have grown based on biophysical parameters related to biomass of the entire plant and estimated from reflectance data and on environmental conditions including solar radiation. In accordance with one embodiment, the dry weight biomass of the entire plant is calculated as:

W=ΣⱼiPARⱼ*RUE Eq. 10

where W is the dry weight biomass of the entire plant on a per area basis and RUE is the radiation use efficiency which is a function of climate conditions, crop species, crop variety and crop nitrogen status.

In other embodiments, the dry weight biomass is computed directly from the Leaf Area Index without computing the intercepted photosynthetically active radiation first as:

W=f(LAIⱼ) Eq. 11

In still further embodiments, the dry weight biomass is computed directly from reflectance data 334 without performing any of steps 402-410 as:

W=f(VI, Reflectance) Eq. 12

where Reflectance represents a subset of reflectance measurements collected from the multiple spectral bands and VI represents a subset of vegetative indices able to be calculated using a given subset of spectral bands collected for a given set of imagery in reflectance data.

Once the dry weight biomass has been determined at step 212 of FIG. 2, the critical nitrogen concentration for the aboveground vegetative portions of the plants (N_{c,v}) is determined at step 213 by the processor of computer 304 using a Critical aboveground vegetative Nitrogen Dilution Curve (CvNDC), which is defined as:

N_{c,v}=aᵥW^{-b}^{ᵥ} Eq. 13

where W is the dry weight biomass of the entire plant determined using one of equations 10-12 and aᵥ and b_{y} are parameters defining the relationship between the dry weight biomass of the entire plant and the critical nitrogen concentration for the aboveground vegetative portions of the plant. An example of such a relationship is shown as graph 110 in FIG. 1

At step 214, the processor executes a nitrogen concentration prediction module 354 to estimate the actual amount of nitrogen in the area of the field using the reflectance data. In accordance with one embodiment, the estimated actual nitrogen concentration is only the nitrogen concentration for the aboveground vegetative portion of the plant since that is all that is visible to the remote sensing. In such embodiments, the estimated aboveground nitrogen concentration is computed as:

% N_{a,v}=f(VI, Reflectance) Eq. 14

where N_{a,v} is the estimated actual aboveground vegetative nitrogen concentration, Reflectance represents a subset of reflectance measurements collected from the multiple spectral bands and VI represents a subset of vegetative indices able to be calculated using a given subset of spectral bands collected for a given set of imagery in reflectance data.

In accordance with some embodiments, a partial least squares regression model is used that is based on a plurality of different vegetation indices calculated using a plurality of spectral bands. In accordance with one particular embodiment, thirty-one vegetation indices computed from twenty-six spectral bands are used in the partial least squares regression model.

At step 215, the estimated aboveground vegetative nitrogen concentration is used by the processor to form a nitrogen nutrition index for the aboveground vegetative portions of the plants (NNIᵥ). The NNI_{V} is computed as:

$${NNI}_{v} = \frac{{N}_{a , v}}{{N}_{c , v}} \qquad Eq . 15$$

The nitrogen nutrition index for the aboveground vegetative portions is then converted to an estimate of the nitrogen nutrition index for the entire plant as:

NNI=Cₜ*NNIᵥ Eq. 16

where NNI is the nitrogen nutrition index for the entire plant, NNIᵥ is the nitrogen nutrition index for the aboveground vegetative portions of the plant and Cₜ is time-dependent coefficient that varies based on the time in the growing season.

At step 216, the processor executes a crop Nitrogen status module 357 to compute the current nitrogen status of the area of the field. In accordance with one embodiment, the current nitrogen status is computed as:

CNS=N_{c,u}(NNI−NNI_{Opt}) Eq. 17

where CNS is the current crop nitrogen status in terms of mass of nitrogen per area, NNI is the nitrogen nutrition index for the entire plant computed above and NNI_{Opt} is the optimal nitrogen nutrition index 358, which is provided to image processing computer 304 based on the crop species, cultivar and environmental conditions and is stored in the memory of computer 304. Typically, NNI_{Opt} is equal to 1 such that the actual nitrogen concentration is equal to the critical nitrogen concentration N, but could vary based on crop species, cultivar or environmental conditions.

Nc,u in equation 17 is computed as:

N_{c,u}=a′W^{(1-b)}=10 a W^{(1-b)} Eq. 18

where a′ is equal to 10a, and a and b are parameters of the CNDC relationship between dry weight biomass W of the entire plant and the critical nitrogen concentration for the entire plant.

In Equation 17, NNI _{Opt} is the optimal NNI for the entire plant and as such is the ratio of the optimal measured nitrogen concentration over the critical nitrogen concentration for the entire plant. NNI is the ratio of the actual measured nitrogen concentration for the entire plant over the critical nitrogen concentration for the entire plant. N_{c,u} represents the critical nitrogen concentration for the entire plant as predicted by CNDC (aW^{-b}, Eq. 1 above) times the dry weight biomass W of the entire plant times a factor of 10. Multiplying the critical nitrogen concentration for the entire plant by the dry weight biomass of the entire plant provides an amount of nitrogen that is required at a given point in the growing season to maximize the relative rate of crop growth. The factor of 10 accounts for the dry weight biomass of the entire plant being expressed on a mass per area basis in units of megagrams per hectare, while the critical nitrogen concentration is expressed on a mass per mass basis in units of grams nitrogen per 100 grams, and the critical nitrogen content is expressed on a mass per area basis in units of kilograms nitrogen per hectare.

The success of Equation 17 in estimating the current nitrogen status of the area of the field is dependent on the inventor's discovery that the nitrogen nutrition index for the aboveground vegetative portion of the plant (NNI_{V}) can be converted into a nitrogen nutritional index for the entire plant.

In accordance with one embodiment, the crop nitrogen status of Equation 17 can be used directly to determine whether the area has an optimum amount of nitrogen and to adjust the rate of nitrogen applied to the field. If the CNS is less than 0, the magnitude of CNS is the rate at which nitrogen should be applied in kilograms of nitrogen per hectare to the field to achieve optimum growth. If the CNS is positive, it indicates an excessive rate of nitrogen that has been applied to the field in kilograms per hectare and provides an indication of how the amount of applied nitrogen can be reduced in future years or an indication of potential negative environmental impact.

In other embodiments, the CNS is not used directly but instead is adjusted to account for nitrogen uptake efficiency, which is the ratio of plant nitrogen uptake to the total of all nitrogen inputs applied to the plant. In most cases, the N uptake efficiency (NUpE) is less 1. To account for this, the CNS is adjusted as:

N_{Fertilzier}=CNS/NUpE Eq. 19

where N_{Fertilizer} is the rate at which fertilizer is to be applied to the field.

At step 218, the current crop nitrogen status is stored and at step 224, the current crop nitrogen status 349 is output to a user. This output can be in the form of a map of the field showing the current nitrogen status of the area of the field using, for example, color coding.

At step 220, past crop nitrogen statuses for the area of the field are retrieved and at step 222 these past crop nitrogen statuses are integrated to create a weighted sum of crop nitrogen statuses. This weighted sum represents a cumulative or integrated crop nitrogen status for the area of the field so far during the growing season and as such represents an aggregated version of the nitrogen status that considers previous conditions and their effect on cumulative crop growth. At step 226, the integrated nitrogen status is also output using for example, color coding on a map.

In accordance with some embodiments, a future biomass prediction module 362 estimates a future biomass 364 at step 230 based on the current biomass, expected weather conditions until the end of the growing season, and a growth model based on the expected weather conditions. In accordance with one embodiment, the future biomass 364 is output at step 232 as a color coding on a map.

At step 232, a harvest index prediction module 366 estimates a harvest index as:

HI=f(crop species, crop variety, time, Crop N Status) Eq. 20

where HI is the harvest index and Crop N Status is the integrated crop nitrogen status.

At step 236, a yield prediction module 368 estimates a yield from the predicted future biomass and the predicted harvest index as:

Yield=W*HI Eq. 21

where W is the estimated future biomass for the entire plant.

At step 238, yield prediction module 368 outputs the estimated yield 370. In accordance with one embodiment, estimated yield 370 is output as a color coding on a map.

The process of FIG. 2 is repeated over the course of the growing season and is performed for multiple areas in the field and for multiple fields to indicate the nitrogen status across a farming operation. In accordance with some embodiments, the output of the current nitrogen status 224 is performed simultaneously for all areas of a field or for all areas of a farming operation to show a comparison of the nitrogen levels of the different areas of the operation. In accordance with some embodiments, the output of the integrated nitrogen status 226 is performed simultaneously for all areas of a field or for all areas of a farming operation to show a comparison of the nitrogen levels of the different areas of the operation.

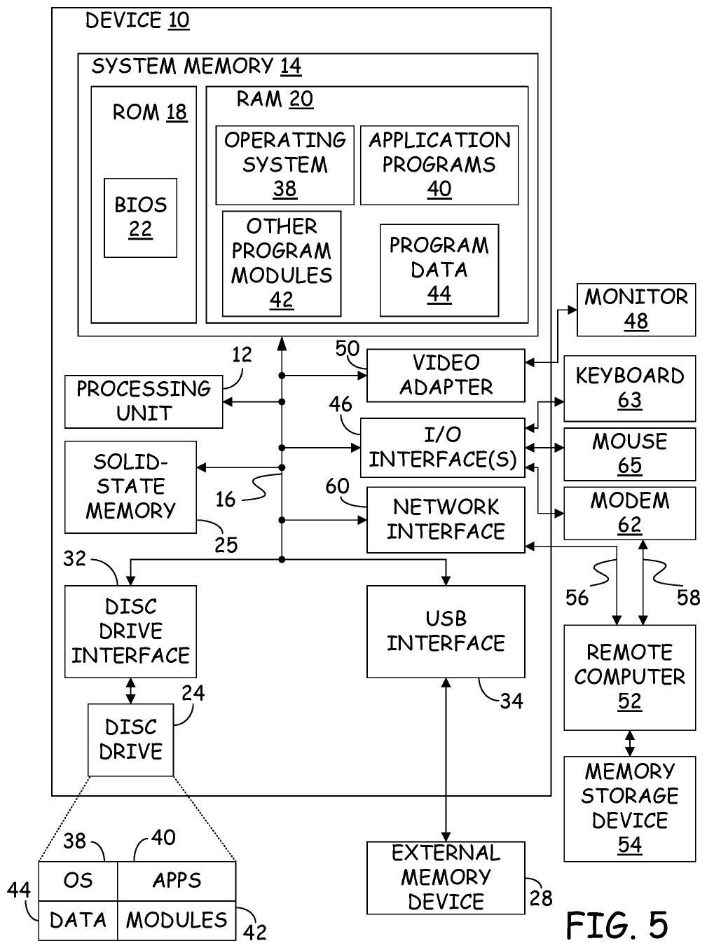

FIG. 5 provides an example of a computing device 10 that can be used as a server or client device in the embodiments above. Computing device 10 includes a processing unit 12, a system memory 14 and a system bus 16 that couples the system memory 14 to the processing unit 12. System memory 14 includes read only memory (ROM) 18 and random access memory (RAM) 20. A basic input/output system 22 (BIOS), containing the basic routines that help to transfer information between elements within the computing device 10, is stored in ROM 18. Computer-executable instructions that are to be executed by processing unit 12 may be stored in random access memory 20 before being executed.

Embodiments of the present invention can be applied in the context of computer systems other than computing device 10. Other appropriate computer systems include handheld devices, multi-processor systems, various consumer electronic devices, mainframe computers, and the like. Those skilled in the art will also appreciate that embodiments can also be applied within computer systems wherein tasks are performed by remote processing devices that are linked through a communications network (e.g., communication utilizing Internet or web-based software systems). For example, program modules may be located in either local or remote memory storage devices or simultaneously in both local and remote memory storage devices. Similarly, any storage of data associated with embodiments of the present invention may be accomplished utilizing either local or remote storage devices, or simultaneously utilizing both local and remote storage devices.

Computing device 10 further includes an optional hard disc drive 24 and an optional external memory device 28. External memory device 28 can include an external disc drive or solid state memory that may be attached to computing device 10 through an interface such as Universal Serial Bus interface 34, which is connected to system bus 16. Hard disc drive 24 is connected to the system bus 16 by a hard disc drive interface 32. The drives and external memory devices and their associated computer-readable media provide nonvolatile storage media for the computing device 10 on which computer-executable instructions and computer-readable data structures may be stored. Other types of media that are readable by a computer may also be used in the exemplary operation environment.

A number of program modules may be stored in the drives and RAM 20, including an operating system 38, one or more application programs 40, other program modules 42 and program data 44. In particular, application programs 40 can include programs for implementing any one of modules discussed above. Program data 44 may include any data used by the systems and methods discussed above.

Processing unit 12, also referred to as a processor, executes programs in system memory 14 and solid state memory 25 to perform the methods described above.

Input devices including a keyboard 63 and a mouse 65 are optionally connected to system bus 16 through an Input/Output interface 46 that is coupled to system bus 16. Monitor or display 48 is connected to the system bus 16 through a video adapter 50 and provides graphical images to users. Other peripheral output devices (e.g., speakers or printers) could also be included but have not been illustrated. In accordance with some embodiments, monitor 48 comprises a touch screen that both displays input and provides locations on the screen where the user is contacting the screen.

The computing device 10 may operate in a network environment utilizing connections to one or more remote computers, such as a remote computer 52. The remote computer 52 may be a server, a router, a peer device, or other common network node. Remote computer 52 may include many or all of the features and elements described in relation to computing device 10, although only a memory storage device 54 has been illustrated in FIG. 5. The network connections depicted in FIG. 5 include a local area network (LAN) 56 and a wide area network (WAN) 58. Such network environments are commonplace in the art.

The computing device 10 is connected to the LAN 56 through a network interface 60. The computing device 10 is also connected to WAN 58 and includes a modem 62 for establishing communications over the WAN 58. The modem 62, which may be internal or external, is connected to the system bus 16 via the I/O interface 46.

In a networked environment, program modules depicted relative to the computing device 10, or portions thereof, may be stored in the remote memory storage device 54. For example, application programs may be stored utilizing memory storage device 54. In addition, data associated with an application program may illustratively be stored within memory storage device 54. It will be appreciated that the network connections shown in FIG. 5 are exemplary and other means for establishing a communications link between the computers, such as a wireless interface communications link, may be used.

Although the present invention has been described with reference to preferred embodiments, workers skilled in the art will recognize that changes may be made in form and detail without departing from the spirit and scope of the invention.

What is claimed is:

1. A method of determining the nitrogen status of an area of land, the method comprising:
determining a critical nitrogen concentration for aboveground vegetation of plants in the area of land based on a dry weight biomass of entire plants;
determining an actual nitrogen concentration for the aboveground vegetation of the plants;
determining a critical nitrogen concentration for the entire plants based on the dry weight biomass of the entire plants; and
combining the actual nitrogen concentration for the aboveground vegetation, the critical nitrogen concentration for the aboveground vegetation, the critical nitrogen concentration for the entire plants and the dry weight biomass of the entire plants to form the nitrogen status for the area of land.

2. The method of claim 1 further comprising estimating the dry weight biomass based on reflectance data for the area of land.

3. The method of claim 1 wherein the actual nitrogen concentration for the aboveground vegetation is estimated from reflectance data for the area of land.

4. The method of claim 1 wherein combining the actual nitrogen concentration for the aboveground vegetation and the critical nitrogen concentration for the aboveground vegetation comprises forming a nitrogen index as a ratio of the actual nitrogen concentration for the aboveground vegetation over the critical nitrogen concentration for the aboveground vegetation.

5. The method of claim 4 further comprising determining a difference between the nitrogen index and an optimal nitrogen index.

6. The method of claim 5 wherein combining further comprises multiplying the dry weight biomass, the critical nitrogen concentration for the entire plants and the difference between the nitrogen index and the optimal nitrogen index.

7. The method of claim 1 further comprising determine the nitrogen status for the area of land on a plurality of days and combining the nitrogen status for the plurality of days to form an integrated nitrogen status for the area of land.

8. A computer comprising:
a memory storing reflectance data for an area of a field; and
a processor executing instructions to perform steps comprising:
using the reflectance data to determine an estimated nitrogen concentration for aboveground vegetation in the area;
using the estimated nitrogen concentration for aboveground vegetation in the area to determine a rate of nitrogen application needed by the area.

9. The computer of claim 8 wherein the processor performs further steps comprising using the reflectance data to determine a dry weight biomass for entire plants in the area.

10. The computer of claim 9 wherein determining a rate of nitrogen application needed by the area comprises using the dry weight biomass to determine the rate of nitrogen application needed by the area.

11. The computer of claim 10 wherein using the estimated nitrogen concentration for aboveground vegetation in the area comprises forming a ratio of the estimated nitrogen concentration for aboveground vegetation in the area to a critical nitrogen concentration for aboveground vegetation in the area to form a nitrogen index.

12. The computer of claim 10 wherein determining a rate of nitrogen application needed by the area further comprises determining the critical nitrogen concentration for aboveground vegetation in the area using the dry weight biomass.

13. The computer of claim 12 wherein determining a rate of nitrogen application needed by the area further comprises determining a difference between the nitrogen index and an optimal nitrogen index.

14. The computer of claim 13 wherein determining a rate of nitrogen application needed by the area further comprises determining a critical nitrogen concentration for entire plants and multiplying the critical nitrogen concentration for entire plants by the difference.

15. A method comprising:
receiving reflectance data for an area of a field;
using the reflectance data to determine an estimated nitrogen concentration for aboveground vegetation in the area; and
using the estimated nitrogen concentration for aboveground vegetation to determine whether the area has an optimum amount of nitrogen.

16. The method of claim 15 wherein using the estimated nitrogen concentration for aboveground vegetation to determine whether the area has the optimum amount of nitrogen comprises forming a ratio of the estimated nitrogen concentration for aboveground vegetation to a critical nitrogen concentration for aboveground vegetation to form a nitrogen index and using the nitrogen index to determine whether the area has the optimum amount of nitrogen.

17. The method of claim 16 wherein using the nitrogen index to determine whether the area has the optimum amount of nitrogen comprises determining a difference between the nitrogen index and an optimum nitrogen index.

18. The method of claim 17 wherein determining whether the area has the optimum amount of nitrogen comprises multiplying the difference by the dry weight biomass of entire plants in the area.

19. The method of claim 18 further comprising estimating the dry weight biomass from the reflectance data for the area of the field.

20. The method of claim 18 wherein determining whether the area has the optimum amount of nitrogen comprises determining an integrated crop nitrogen status.

21. The method of claim 20 further comprising predicting a yield from the integrated crop nitrogen status.

22. The method of claim 18 wherein determining whether the area has the optimum amount of nitrogen comprises estimating potential environmental impact.
